(19) United States
(12) Patent Application Publication
(10) Pub. No.: US 2022/0241266 A1
(43) Pub. Date: Aug. 4, 2022

(54) COMPOSITIONS AND METHODS USING TRIGONELLINE AND HIGH PROTEIN FOR PREVENTING OR TREATING CONDITIONS OR DISORDERS IN SKELETAL MUSCLE

(71) Applicant: SOCIETE DES PRODUITS NESTLE S.A., Vevey (CH)

(72) Inventors: JEROME FEIGE, Boca Raton, FL (US); MATHIEU MEMBREZ, Cugy (CH); VINCENZO SORRENTINO, Lausanne (CH); STEFAN CHRISTEN, Ecublens (CH); MARIA PILAR GINER, Pully (CH); SOFIA MOCO, Lausanne (CH)

(21) Appl. No.: 17/597,343

(22) PCT Filed: Jul. 3, 2020
(86) PCT No.: PCT/EP2020/068773

§ 371 Date: Jan. 4, 2022

(30) Foreign Application Priority Data
Jul. 5, 2019 (EP) 19184815.9

(51) Int. Cl. A61K 31/455; A61K 31/405; A61K 38/01; A61P 21/00; A61K 36/48; A61K 31/198; A23L 33/105; A23L 33/19

(52) U.S. Cl. CPC A61K 31/455; A61K 31/405; A61K 38/011; A61K 38/018; A23L 33/19; A61K 36/48; A61K 31/198; A23L 33/105; A61P 21/00

(57) ABSTRACT

The present invention relates to compositions and methods for preventing or treating skeletal muscle conditions or diseases. The present invention also relates to compositions and methods that help to increase levels of NAD+ in skeletal muscle. Preferably, the present invention relates to compositions and methods that use trigonelline, high protein and/or creatine and/or tryptophan for preventing or treating skeletal muscle conditions or diseases. The recipient of the compositions or methods of the invention can be, for example, an elderly individual or an individual with sarcopenia or an individual in need of the compositions and methods of the invention for recovery of skeletal muscles, for example, after exercise, muscle injury or surgery.

### BACKGROUND OF THE INVENTION

Age-related loss of muscle mass and function is inevitable in all individuals, however, its progression largely depends on genetic and environmental factors such as physical activity and nutritional intake, in particular adequate intake of protein. Sarcopenia has been defined as the point where the age-related loss of muscle mass and function gets debilitating and impacts quality of life. In contrast, frailty is another classification of age-related physical function decline that features low muscle strength and functionality, but not muscle mass. Sarcopenia is defined clinically according to low muscle mass and function, using cutoffs which stratify the elderly population for individuals in a state of pathological mobility. Sarcopenia predicts future disability and mortality, and was assigned an official ICD-10 disease code in 2016 (Anker et al., J Cachexia Sarcopenia Muscle, 2016 December; 7(5): 512-514).

Nicotinic acid (also known as niacin) and nicotinamide, both forms of vitamin B3 are the precursors of nicotinamide adenine dinucleotide (NAD⁺). Eukaryotes can synthesize NAD⁺ de novo via the kynurenine pathway from tryptophan, and niacin supplementation prevents the pellagra that can occur in populations with a tryptophan-poor diet. Nicotinic acid is phosphoribosylated to nicotinic acid mononucleotide (NaMN), which is then adenylated to form nicotinic acid adenine dinucleotide (NaAD), which in turn is amidated to form NAD⁺.

NAD⁺ is an enzyme co-factor that is essential for the function of several enzymes related to reduction-oxidation reactions and energy metabolism. NAD⁺ functions as an electron carrier in cell metabolism of amino acids, fatty acids, and carbohydrates. NAD⁺ also serves as an activator and substrate for sirtuins, a family of protein deacetylases that have been implicated in metabolic function and extended lifespan in lower organisms. The co-enzymatic activity of NAD⁺, together with the tight regulation of its biosynthesis and bioavailability, makes it important in the metabolic monitoring system and also in the aging process.

Trigonelline is a niacin (Vitamin B3) metabolite, formed by the methylation of the nitrogen atom of niacin, and is excreted in urine of mammals. As a bioactive molecule, it has been isolated from extracts found in a number of plant and algae species.

In various skeletal muscle conditions and diseases where NAD+ needs to be boosted, the present invention provides solutions for preventing and/or treating these skeletal muscle conditions and diseases.

### SUMMARY OF THE INVENTION

The present disclosure provides a composition consisting essentially of trigonelline or consisting essentially of trigonelline and high protein.

In some embodiments, a composition is provided consisting essentially of trigonelline, high protein and creatine.

In some embodiments, a composition is provided consisting essentially of trigonelline, high protein, creatine and/or tryptophan.

In some embodiments, at least a portion of the trigonelline is provided from a plant source by a plant extract in the composition, such as one or more of a coffee extract, a hemp extract, a pumpkin seed extract and/or a fenugreek extract, for example, a plant extract enriched in trigonelline.

In a preferred embodiment, at least a portion of trigonelline is provided from a fenugreek extract.

In some embodiments, at least a portion of the trigonelline is provided from an algae source, for example, a Laminariaceae extract.

In some embodiments, the high protein is provided by plant source.

In some embodiments, the protein can comprise peptides having a length of 2 to 10 amino acids.

In some embodiments, the amino acids are branched chain amino acids such as leucine, isoleucine and/or valine.

In one embodiment, the amino acid is 4-hydroxyisoleucine.

In some embodiments, the amino acid tryptophan is provided in the composition.

In some embodiments, creatine is provided in the composition.

In an embodiment, the composition formulation is selected from the group consisting of: a food product, beverage product, a food supplement, an oral nutritional supplement (ONS), a medical food, and combinations thereof.

The increase in NAD⁺ biosynthesis can provide one or more benefits to the individual, for example in a human (e.g., a human undergoing medical treatment), in an animal such as a dog, cat, cow, horse, pig, or sheep (e.g., a companion animal such as a dog or cat undergoing medical treatment), or in cattle, poultry, swine, ovine (e.g., used in agriculture for milk or meat production).

Preferably, the NAD⁺ biosynthesis is increased in one or more cells of the mammal, for example one or more cells that are part of the musculoskeletal system such as skeletal muscle.

In an embodiment, the composition is administered enterally.

In one embodiment, the present invention provides a unit dosage form of a composition consisting essentially of trigonelline or consisting of trigonelline. The unit dosage form contains an effective amount of the composition of the invention to treat or prevent (e.g., reducing incidence and/or severity) a disease or a condition associated with NAD+ in an individual in need thereof or at risk thereof by increasing levels of nicotinamide adenine dinucleotide (NAD+) in cells and tissues to improve cell and tissue survival and/or or overall cell and tissue health, in particular, in skeletal muscle.

In another embodiment, the present invention provides a unit dosage form of a composition consisting essentially of trigonelline and high protein or consisting of trigonelline and high protein. The unit dosage form contains an effective amount of the composition to treat or prevent (e.g., reducing incidence and/or severity) a disease or a condition associated with associated with NAD+ in an individual in need thereof or at risk thereof by increasing levels of nicotinamide adenine dinucleotide (NAD+) in cells and tissues to improve cell and tissue survival and/or or overall cell and tissue health, in particular, in skeletal muscle.

In another embodiment, the present invention provides a unit dosage form of a composition consisting essentially of trigonelline, high protein and creatine or consisting of trigonelline, high protein, and creatine. The unit dosage form contains an effective amount of the composition to treat or prevent (e.g., reducing incidence and/or severity) a disease or a condition associated with NAD+ in an individual in need thereof or at risk thereof by increasing levels of nicotinamide adenine dinucleotide (NAD+) in cells and tissues to improve cell and tissue survival and/or or overall cell and tissue health, in particular, in skeletal muscle.

In another embodiment, the present invention provides a unit dosage form of a composition consisting essentially of trigonelline, high protein, creatine and/or tryptophan or consisting of trigonelline, high protein, creatine and/or tryptophan. The unit dosage form contains an effective amount of the composition to treat or prevent (e.g., reducing incidence and/or severity) a disease or a condition associated with NAD+ in an individual in need thereof or at risk thereof by increasing levels of nicotinamide adenine dinucleotide (NAD+) in cells and tissues to improve cell and tissue survival and/or or overall cell and tissue health, in particular, in skeletal muscle.

The composition can be selected from the group consisting of: a food product, a beverage product, a food supplement, an oral nutritional supplement (ONS), a medical food, and combinations thereof.

One advantage of one or more embodiments provided by the present invention is to replenish NAD⁺ pools, which decline with age.

Another advantage of one or more embodiments provided by the present invention is to help off-set slowing of the metabolism associated with aging.

An advantage of one or more embodiments provided by the present invention is to potentiate benefits on oxidative metabolism and prevent DNA damage.

Yet another advantage of one or more embodiments provided by the present invention is to help the body to metabolize fat and increase lean body mass.

Another advantage of one or more embodiments provided by the present invention is to maintain or increase skeletal muscle function in a subject.

Another advantage of one or more embodiments provided by the present invention is to increase muscle function, for example, by an increase in the number of muscle stem cells and/or myoblasts and/or myotubes.

Another advantage of one or more embodiments provided by the present invention is to maintain or increase skeletal muscle mass in a subject.

Another advantage of one or more embodiments provided by the present invention is to prevent or reduce skeletal muscle wasting in a subject.

Another advantage of one or more embodiments provided by the present invention is to enhance recovery of skeletal muscle after intense exercise.

Another advantage of one or more embodiments provided by the present invention is to enhance recovery of skeletal muscle after injury.

Another advantage of one or more embodiments provided by the present invention is to enhance recovery of skeletal muscle after trauma or surgery.

Yet another advantage of one or more embodiments provided by the present invention is to support improvements, as mentioned above, in the skeletal muscle after diseases and conditions such as: cachexia or precachexia; sarcopenia, myopathy, dystrophy, and/or recovery after intense exercise, muscle injury or surgery. In particular, cachexia is associated with cancer, chronic heart failure, renal failure, chronic obstructive pulmonary disease, AIDS, autoimmune disorders, chronic inflammatory disorders, cirrhosis of the liver, anorexia, chronic pancreatitis, metabolic acidosis and/or neurodegenerative disease.

In one embodiment, the invention provides a method for increasing NAD+ in a subject mammal comprising delivering to the mammal in need of such treatment an effective amount of a composition according to the invention in an effective unit dose form to prevent and/or treat skeletal muscle diseases or conditions. The skeletal muscle disease or condition such as cachexia or precachexia; sarcopenia, myopathy, dystrophy, and/or recovery after intense exercise, muscle injury or surgery.

In another embodiment, the invention provides a method for increasing NAD+ in a subject mammal for preventing and/or treating skeletal muscle disease or conditions in a subject in need comprising the steps of:

i) providing the subject a composition consisting essentially of or consisting of trigonelline and high protein; and
ii) administering the composition to said subject.

In yet another embodiment, the invention provides a method for increasing NAD+ in a subject mammal for preventing and/or treating skeletal muscle disease or conditions in a subject in need comprising the steps of:

i) providing the subject a composition consisting essentially of or consisting of trigonelline, high protein and creatine; and
ii) administering the composition to said subject.

In yet another embodiment, the invention provides a method for increasing NAD+ in a subject mammal for preventing and/or treating skeletal muscle disease or conditions in a subject in need comprising the steps of:

i) providing the subject a composition consisting essentially of or consisting of trigonelline, high protein in the form of branched chain amino acids and creatine; and
ii) administering the composition to said subject.

In yet another embodiment, the invention provides a method for increasing NAD+ in a subject mammal for preventing and/or treating skeletal muscle disease or conditions in a subject in need comprising the steps of:

i) providing the subject a composition consisting essentially of or consisting of trigonelline, high protein, creatine and/or tryptophan; and
ii) administering the composition to said subject.

In some embodiments, the subject includes human, dog, cat, cow, horse, pig, or sheep. In some embodiments, the subject is preferably a human.

### DESCRIPTION OF FIGURES

FIG. 1—Enzymatic quantification of NAD+ concentration in Human and Zebrafish upon trigonelline treatment

FIG. 1A shows the enzymatic quantification of NAD+ concentration in Human Skeletal Muscle Myotubes (HSMM) treated for 6 h with trigonelline in doses 5 μM, 50 μM, 500 μM and 1 mM.

FIG. 1B shows the enzymatic quantification of NAD+ concentration in zebrafish larvae (DPF4) treated for 16 h with trigonelline in doses 500 μM and 1 mM.

#, * indicate difference from the control, One-way ANOVA, with p<0.1, p<0.05 respectively. Data are presented as Mean+/−SEM

FIG. 2—Mass spectrometry NAD+ concentration in Myotubes and Stable Isotope labelled incorporation into NAD+ upon trigonelline treatment

FIG. 2A shows the NAD+ relative concentration in Human Skeletal Muscle Myotubes (HSMM) from 2 different donors treated for 6 h with trigonelline at dose 500 μM relative to control, measured by liquid chromatography-mass spectrometry (LC-MS).

FIG. 2B shows the relative abundance of labelled trigonelline at dose 500 μM incorporated into NAD+(M+1), measured by LC-MS.

**, **** indicate difference from the respective control, unpaired t-test, with p<0.01, p<0.0001, respectively. Data are presented as Mean+/−SEM

FIG. 2C shows the stable isotope labelled incorporation into NAD+ upon trigonelline treatment. C* represents the labelled ¹³C (M+1 over natural ¹²C) and D3 represents deuterium/²H (M+1 over natural ¹H).

FIG. 3—Enzymatic quantification of NAD+ uptake in Liver and Muscle upon trigonelline treatment

Enzymatic quantification of NAD+ in mice 120 minutes after receiving 250 mg/kg trigonelline by oral gavage (FIGS. 3A, 3C) or intraperitoneal administration (FIGS. 3B, 3D).

* indicates difference from the control, unpaired t-test with p<0.05. Data are presented as Mean+/−SEM

FIG. 4—NAD⁺ measured in human primary myoplasts after treatment of chemically synthesized trigonelline or fenugreek seed extract enriched in trigonelline

FIG. 4A shows Human Skeletal Muscle Myotubes (HSMM) treated for 16 h with synthetic trigonelline monohydrate at different doses and quantification of NAD

FIG. 4B shows Human Skeletal Muscle Myotubes (HSMM) treated for 16 h with a fenugreek seed extract enriched in trigonelline (40.45% trigonelline) at different doses and quantification of NAD

*, **, **** indicate difference from the control, One-way ANOVA, with p<0.05, p<0.01, p<0.001, respectively. Data are presented as Mean+/−SD

FIG. 5—Liver NAD⁺ levels of C57BL/6JRj mice measured 120 minutes after administration of 300 mg/kg trigonelline chloride or an equimolar amount of fenugreek seed extract by oral gavage

*, **, **** indicate difference from the control, One-way ANOVA, with p<0.05, p<0.01, p<0.001, respectively. Data are presented as Mean+/−SD

FIG. 6—C. elegans whole-lysate NAD⁺ levels measured in Day 1 adult animals, and in Day 8 aged worms treated with 1 mM trigonelline chloride, compared to their age-matched controls

*, **, **** indicate difference from the control, One-way ANOVA, with p<0.05, p<0.01, p<0.001, respectively. Data are presented as Mean+/−SD

FIG. 7—C. elegans survival, mean speed, distance and mobility

FIG. 7A—Survival curve of C. elegans treated with 1 mM trigonelline chloride increases lifespan by 21%.

FIG. 7B—Mean speed measured during spontaneous mobility assay performed from day 1 adulthood in 1 mM trigonelline chloride treated worms compared to controls.

FIG. 7C—Distance travelled during the spontaneous mobility assay in advanced aging phase.

FIG. 7D Stimulated mobility score assessed for day 8 and day 11 old worms indicate the percentage of worms responsive to a physical stimulus.

*,** indicate difference from the control, Student test, with p<0.05, p<0.01, respectively.

For FIGS. 7A & D, data are presented as Mean+/−SD.

For FIGS. 7B & C, data are presented as Mean+/−SEM.

FIG. 8—C. elegans mitochondrial to nuclear DNA ratio (mt/nDNA)

FIG. 8 shows the ratio of a mitochondrial-encoded gene (nduo-1) represented as relative to a nuclear-encoded gene (act-1) in day 8 old worms.

*indicate difference from the control, Student test, with p<0.05.

Data are presented as Mean+/−SD

### DETAILED DESCRIPTION OF THE INVENTION

### Definitions

All percentages are by weight of the total weight of the composition unless expressed otherwise. Similarly, all ratios are by weight unless expressed otherwise. When reference is made to the pH, values correspond to pH measured at 25° C. with standard equipment. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used herein and in the appended claims, the singular form of a word includes the plural, unless the context clearly dictates otherwise. Thus, the references “a,” “an” and “the” are generally inclusive of the plurals of the respective terms. For example, reference to “an ingredient” or “a method” includes a plurality of such “ingredients” or “methods.” The term “and/or” used in the context of “X and/or Y” should be interpreted as “X,” or “Y,” or “X and Y.” Similarly, “at least one of X or Y” should be interpreted as “X,” or “Y,” or “both X and Y.”

Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of” and “consisting of” the disclosed components. “Consisting essentially of” means that the embodiment comprises more than 50 wt. % of the identified components, preferably at least 75 wt. % of the identified components, more preferably at least 85 wt. % of the identified components, most preferably at least 95 wt. % of the identified components, for example at least 99 wt. % of the identified components.

Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

“Animal” includes, but is not limited to, mammals, which includes but is not limited to rodents, aquatic mammals, domestic animals such as dogs and cats, farm animals such as sheep, pigs, cows and horses, and humans. Where “animal,” “mammal” or a plural thereof is used, these terms also apply to any animal that is capable of the effect exhibited or intended to be exhibited by the context of the passage, e.g., an animal capable of autophagy. As used herein, the term “subject” or “patient” is understood to include an animal, for example a mammal, and preferably a human that is receiving or intended to receive treatment, as treatment is herein defined. While the terms “individual” and “patient” are often used herein to refer to a human, the present disclosure is not so limited.

Accordingly, the terms “subject”, “individual” and “patient” refer to any animal, mammal or human that can benefit from the methods and compositions disclosed herein. Indeed, non-human animals undergo prolonged critical illness that mimics the human condition. These critically ill animals undergo the same metabolic, immunological and endocrine disturbances and development of organ failure and muscle wasting as the human counterpart. Moreover, animals experience the effects of ageing as well.

The term “elderly” in the context of a human means an age from birth of at least 55 years, preferably above 63 years, more preferably above 65 years, and most preferably above 70 years. The term “older adult” or “ageing individual” in the context of a human means an age from birth of at least 45 years, preferably above 50 years, more preferably above 55 years, and includes elderly individuals.

For other animals, an “older adult” or “ageing individual” has exceeded 50% of the average lifespan for its particular species and/or breed within a species. An animal is considered “elderly” if it has surpassed 66% of the average expected lifespan, preferably if it has surpassed the 75% of the average expected lifespan, more preferably if it has surpassed 80% of the average expected lifespan. An ageing cat or dog has an age from birth of at least about 5 years. An elderly cat or dog has an age from birth of at least about 7 years.

### Sarcopenia

“Sarcopenia” is defined as the age-associated loss of muscle mass and functionality (including muscle strength and gait speed). Sarcopenia can be characterized by one or more of low muscle mass, low muscle strength and low physical performance.

Sarcopenia can be diagnosed in a subject based on the definition of the AWGSOP (Asian Working Group for Sarcopenia in Older People), for example as described in Chen et al., 2014 J Am Med Dir Assoc. 2014 February; 15(2):95-101. Low muscle mass can generally be based on low appendicular lean mass normalized to height square (ALM index), particularly ALM index less than 7.00 kg/m2 for men and 5.40 kg/m2 for women. Low physical performance can generally be based on gait speed, particularly gait speed of <0.8 m/sec. Low muscle strength can generally be based on low hand grip strength, particularly hand grip strength less than 26 kg in men and less than 18 kg in women.

Additionally or alternatively, sarcopenia can be diagnosed in a subject based on the definition of the EWGSOP (European Working Group for Sarcopenia in Older People), for example as described in Crutz-Jentoft et al., 2019. Age Ageing. 2019 Jan. 1; 48(1):16-31. Low muscle mass can generally be based on low appendicular lean mass normalized to height square (ALM index), particularly ALM index less than 7.23 kg/m2 for men and 5.67 kg/m2 for women. Low physical performance can generally be based on gait speed, particularly gait speed of <0.8 m/sec. Low muscle strength can generally be based on low hand grip strength, particularly hand grip strength less than 30 kg in men and less than 20 kg in women. Additionally or alternatively, sarcopenia can be diagnosed in a subject based on the definition of the Foundation for the National Institutes of Health (FNIH), for example as described in Studenski et al., 2014 J Gerontol A Biol Sci Med Sci. 2014 May; 69(5):547-58. Low muscle mass can generally be based on low appendicular lean mass (ALM) normalized to body mass index (BMI; kg/m2), particularly ALM to BMI less than 0.789 for men and 0.512 for women. Low physical performance can generally be based on gait speed, particularly gait speed of <0.8 m/sec. Low muscle strength can generally be based on low hand grip strength, particularly hand grip strength less than 26 kg in men and less than 16 kg in women. Low muscle strength can also generally be based on low hand grip strength to body mass index, particularly hand grip strength to body mass index less than 1.00 in men and less than 0.56 in women.

As used herein, “frailty” is defined as a clinically recognizable state of increased vulnerability resulting from aging-associated decline in reserve and function across multiple physiologic systems such that the ability to cope with everyday or acute stressors is compromised. In the absence of an established quantitative standard, frailty has been operationally defined by Fried et al. as meeting three out of five phenotypic criteria indicating compromised energetics: (1) weakness (grip strength in the lowest 20% of population at baseline, adjusted for gender and body mass index), (2) poor endurance and energy (self-reported exhaustion associated with VO2 max), (3) slowness (lowest 20% of population at baseline, based on time to walk 15 feet, adjusting for gender and standing height), (4) low physical activity (weighted score of kilocalories expended per week at baseline, lowest quintile of physical activity identified for each gender; e.g., less than 383 kcal/week for males and less than 270 kcal/week for females), and/or unintentional weight loss (10 lbs. in past year). Fried L P, et al., J. Gerontol. A. Biol. Sci. Med. Sci. 56(3):M146-M156 (2001). A pre-frail stage, in which one or two of these criteria are present, identifies a high risk of progressing to frailty.

### Cachexia and Related Diseases

Cachexia is a complex metabolic syndrome associated with underlying illness and characterized by loss of muscle with or without loss of fat mass. The prominent clinical feature of cachexia is weight loss in adults (corrected for fluid retention) or growth failure in children (excluding endocrine disorders).

Cachexia is often seen in patients with diseases such as cancer, chronic heart failure, renal failure, chronic obstructive pulmonary disease, AIDS, autoimmune disorders, chronic inflammatory disorders, cirrhosis of the liver, anorexia, chronic pancreatitis and/or metabolic acidosis and neurodegenerative disease.

There are certain types of cancer wherein cachexia is particularly prevalent, for example, pancreas, esophagus, stomach, bowel, lung and/or liver cancer.

The internationally recognised diagnostic criterion for cachexia is weight loss greater than 5% over a restricted time, for example 6 months, or weight loss greater than 2% in individuals already showing depletion according to current body weight and height (body-mass index [BMI]<20 kg/m²) or skeletal muscle mass (measured by DXA, MRI, CT or bioimpedance). Cachexia can develop progressively through various stages—precachexia to cachexia to refractory cachexia. Severity can be classified according to degree of depletion of energy stores and body protein (BMI) in combination with degree of ongoing weight loss.

In particular, cancer cachexia has been defined as weight loss >5% over past 6 months (in absence of simple starvation); or BMI<20 and any degree of weight loss >2%; or appendicular lean mass consistent with low muscle mass (males <7.26 kg/m²; females <5.45 kg/m²) and any degree of weight loss >2% (Fearon et al. 2011).

Precachexia may be defined as weight loss ≤5% together with anorexia and metabolic change. At present there are no robust biomarkers to identify those precachectic patients who are likely to progress further or the rate at which they will do so. Refractory cachexia is defined essentially on the basis of the patient's clinical characteristics and circumstances.

### Myopathy and Related Conditions

Myopathies are neuromuscular disorders in which the primary symptom is muscle weakness due to dysfunction of muscle fiber. Other symptoms of myopathy can include include muscle cramps, stiffness, and spasm. Myopathies can be inherited (such as the muscular dystrophies) or acquired (such as common muscle cramps).

Myopathies are grouped as follows: (i) congenital myopathies: characterized by developmental delays in motor skills; skeletal and facial abnormalities are occasionally evident at birth (ii) muscular dystrophies: characterized by progressive weakness in voluntary muscles; sometimes evident at birth (iii) mitochondrial myopathies: caused by genetic abnormalities in mitochondria, cellular structures that control energy; include Kearns-Sayre syndrome, MELAS and MERRF glycogen storage diseases of muscle: caused by mutations in genes controlling enzymes that metabolize glycogen and glucose (blood sugar); include Pompe's, Andersen's and Con's diseases (iv) myoglobinurias: caused by disorders in the metabolism of a fuel (myoglobin) necessary for muscle work; include McArdle, Tarui, and DiMauro diseases (v) dermatomyositis: an inflammatory myopathy of skin and muscle (vi) myositis ossificans: characterized by bone growing in muscle tissue (vii) familial periodic paralysis: characterized by episodes of weakness in the arms and legs (viii)polymyositis, inclusion body myositis, and related myopathies: inflammatory myopathies of skeletal muscle (ix) neuromyotonia: characterized by alternating episodes of twitching and stiffness; and stiff-man syndrome: characterized by episodes of rigidity and reflex spasms common muscle cramps and stiffness, and (x) tetany: characterized by prolonged spasms of the arms and legs. (Reference: https://www.ninds.nih.gov/disorders/all-disorders/myopathy-information-page).

Recovery after Muscle Injury from Surgery and Muscle Traumas

Muscle injuries can be caused by bruising, stretching or laceration causing acute or chronic soft tissue injury that occurs to a muscle, tendon, or both. It may occur as a result of fatigue, overuse, or improper use of a muscle. It may occur after physical trauma such as a fall, fracture or overuse during physical activity. Muscle injuries may also occur after surgery such as joint replacement arthroscopic surgery.

The terms “treatment” and “treating” include any effect that results in the improvement of the condition or disorder, for example lessening, reducing, modulating, or eliminating the condition or disorder. The term does not necessarily imply that a subject is treated until total recovery. Non-limiting examples of “treating” or “treatment of” a condition or disorder include: (1) inhibiting the condition or disorder, i.e., arresting the development of the condition or disorder or its clinical symptoms and (2) relieving the condition or disorder, i.e., causing the temporary or permanent regression of the condition or disorder or its clinical symptoms. A treatment can be patient- or doctor-related.

The terms “prevention” or “preventing” mean causing the clinical symptoms of the referenced condition or disorder to not develop in an individual that may be exposed or predisposed to the condition or disorder but does not yet experience or display symptoms of the condition or disorder. The terms “condition” and “disorder” mean any disease, condition, symptom, or indication.

The relative terms “improved,” “increased,” “enhanced” and the like refer to the effects of the composition comprising a combination of trigonelline and high protein (disclosed herein) relative to a composition with less protein but otherwise identical. Likewise the effects of the combination of the composition comprising a combination of trigonelline, high protein and creatine to a composition with less protein but otherwise identical.

The terms “food,” “food product” and “food composition” mean a product or composition that is intended for ingestion by an individual such as a human and provides at least one nutrient to the individual. The compositions of the present disclosure, including the many embodiments described herein, can comprise, consist of, or consist essentially of the essential elements and limitations described herein, as well as any additional or optional ingredients, components, or limitations described herein or otherwise useful in a diet.

The term “beverage”, “beverage product” and “beverage composition” mean a product or composition for ingestion by an individual such as a human and provides at least one nutrient to the individual. The compositions of the present disclosure, including the many embodiments described herein, can comprise, consist of, or consist essentially of the essential elements and limitations described herein, as well as any additional or optional ingredients, components, or limitations described herein or otherwise useful in a diet.

As used herein, “complete nutrition” contains sufficient types and levels of macronutrients (protein, fats and carbohydrates) and micronutrients to be sufficient to be a sole source of nutrition for the subject to which the composition is administered. Individuals can receive 100% of their nutritional requirements from such complete nutritional compositions.

The term “enterally administering” encompasses oral administration (including oral gavage administration), as well as rectal administration, although oral administration is preferred. The term “parenterally administering” refers to delivery of substances given by routes other than the digestive tract and covers administration routes such as intravenous, intra-arterial, intramuscular, intracerebroventricular, intraosseous, intradermal, intrathecal, and also intraperitoneal administration, intravesical infusion and intracavernosal injection.

Preferred parenteral administration is intravenous administration. A particular form of parenteral administration is delivery by intravenous administration of nutrition. Parenteral nutrition is “total parenteral nutrition” when no food is given by other routes. “Parenteral nutrition” is preferably a isotonic or hypertonic aqueous solution (or solid compositions to be dissolved, or liquid concentrates to be diluted to obtain an isotonic or hypertonic solution) comprising a saccharide such as glucose and further comprising one or more of lipids, amino acids, and vitamins.

### Embodiments

### Composition

The present invention comprises a composition comprising a combination of trigonelline and high protein (e.g., at least about 25% of the total energy of the composition), and the composition is administered to provide an amount of the combination that is effective to increase NAD+, for example, in skeletal muscle. The composition can be administered parenterally, enterally, or intravenously.

The present invention comprises a composition consisting essentially of a combination of trigonelline and high protein (e.g., at least about 25% of the total energy of the composition), or consisting essentially of trigonelline, high protein and creatine. A composition of the invention is administered to provide an amount of the combination that is effective to increase NAD+, for example, in skeletal muscle. The composition can be administered parenterally, enterally, or intravenously.

The present invention comprises a composition consisting essentially of a combination of trigonelline and high protein (e.g., at least about 25% of the total energy of the composition) wherein the high protein comprises branched chain amino acids such as leucine, isoleucine and/or valine, or consisting essentially of trigonelline, high protein wherein the high protein consists of branched chain amino acids such as leucine, isoleucine and/or valine. In one preferred embodiment, the high protein is 4-hydroxyisoleucine. A composition of the invention is administered to provide an amount of the combination that is effective to increase NAD+, for example, in skeletal muscle. The composition can be administered parenterally, enterally, or intravenously.

The present invention comprises a composition consisting essentially of a combination of trigonelline and high protein (e.g., at least about 25% of the total energy of the composition) wherein the high protein comprises branched chain amino acids such as leucine, isoleucine and/or valine, and creatine or consists essentially of trigonelline, high protein wherein the high protein consists of branched chain amino acids such as leucine, isoleucine and/or valine and creatine. A composition of the invention is administered to provide an amount of the combination that is effective to increase NAD+, for example, in skeletal muscle. The composition can be administered parenterally, enterally, or intravenously.

In one preferred embodiment, the composition consists essentially of a combination of trigonelline and 4-hydroxyisoleucine.

The present invention comprises a composition consisting essentially of or consisting of a combination of trigonelline and high protein (e.g., at least about 25% of the total energy of the composition), creatine and/or tryptophan. A composition of the invention is administered to provide an amount of the combination that is effective to increase NAD+, for example, in skeletal muscle. The composition can be administered parenterally, enterally, or intravenously.

### Trigonelline

“Trigonelline” is here defined as any compound comprising 1-methylpyridin-1-ium-3-carboxylate including, for example, any salt thereof (e.g., Chloride or Iodide salt) and/or a form in which the ring therein may be reduced.

In some embodiments, trigonelline is represented by the structure of formula 1, being able to establish a salt with an anion (X−), such as a halogen, for example, iodide or chloride. The structure of formula 1 is also known as 3-carboxy-1-methylpyridinium, N-Methylnicotinic acid, 1-methylpyridine-3-carboxylic acid, 1-methylpyridin-1-ium-3-carboxylic acid, Pyridinium 3-carboxy-1-methyl-hydroxide inner salt (8Cl), 1-methylnicotinic acid, Pyridinium 3-carboxy-1-methyl-.

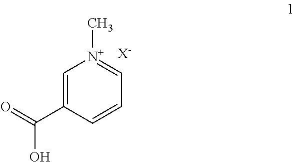

In some embodiments, trigonelline is represented by the structure of formula 2 in its inner salt form. The structure of formula 2 is also known as Caffearine, Gynesine, N-Methylnicotinate, Trigenolline, Coffearine, Trigonellin, Coffearin, Betain nicotinate, Betaine nicotinate, 1-methylpyridinium-3-carboxylate, Nicotinic acid N-methylbetaine, 1-Methylpyridinio-3-carboxylate, 1-Methyl-3-pyridiniumcarboxylate, N-Methylnicotinic acid, Trigenelline, Caffearin, 3-Carboxy-1-methylpyridinium hydroxide inner salt, N′-Methylnicotinate, 1-methylpyridin-1-ium-3-carboxylate, 3-Carboxy-1-methylpyridinium hydroxide inner salt, Pyridinium 3-carboxy-1-methyl-hydroxide inner salt, 1-methylpyridine-3-carboxylic acid, 1-methylpyridin-1-ium-3-carboxylic acid, 1-methylnicotinate, Trigonelline (S), N-methyl-nicotinate, Pyridinium 3-carboxy-1-methyl-hydroxide inner salt (8Cl), N′-Methylnicotinic acid, N-Methylnicotinic acid betaine, Nicotinic acid N-methylbetaine, 1-Methyl-Nicotinic Acid Anion, Pyridinium 3-carboxy-1-methyl-inner salt, 1-Methyl-5-(oxylatocarbonyl)pyridinium-3-ide, Pyridinium 3-carboxy-1-methyl-inner salt, 3-carboxy-1-methyl-Pyridinium hydroxide inner salt).

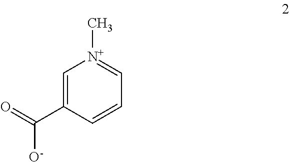

In some embodiments, optionally “trigonelline” can include metabolites and pyrolysis products thereof, such as nicotinamide, nicotinamide riboside, 1-methylnicotinamide, 1-methyl-2-pyridone-5-carboxamide (Me2PY), 1-methyl-4-pyridone-5-carboxamide (Me4PY), and alkyl-pyridiniums, such as 1-methyl-pyridinium (NMP) and 1,4-dimethylpyridinium; although as noted later herein, some embodiments exclude one or more of these metabolites and pyrolysis products of trigonelline.

The composition can comprise a pharmacologically effective amount of trigonelline in a pharmaceutically suitable carrier. In aqueous liquid compositions, the trigonelline concentration preferably ranges from about 0.05 wt. % to about 4 wt. %, or from about 0.5 wt. % to about 2 wt. % or from about 1.0 wt. % to about 1.5 wt. % of the aqueous liquid composition.

In particular embodiments, the method is a treatment that augments the plasma trigonelline for example to a level in the range of 50 to 6000 nmol/L plasma, preferably 100 to 6000 nmol/L plasma. The method can comprise administering daily trigonelline in the weight range of 0.05 mg-1 g per kg body weight, preferably 1 mg-200 mg per kg body weight, more preferably 5 mg-150 mg per kg body weight, even more preferably 10 mg-120 mg per kg body weight, or most preferably 40 mg-80 mg per kg body weight.

Typically between 50 μg to 10 g of trigonelline, per daily serving in one or more portions is administered to a subject. More preferably between 100 mg to 1 g of trigonelline per daily serving in one or more portions is administered to a subject.

In some embodiments, at least a portion of the trigonelline is isolated. Additionally or alternatively, at least a portion of trigonelline can be chemically synthesized.

In one embodiment, the composition comprises trigonelline which is chemically synthesized which is at least about 90% trigonelline, preferably at least about 98% trigonelline.

In a preferred embodiment, at least a portion of the trigonelline is provided by a plant or algae extract, for example an extract from one or more of coffee bean (e.g., a green coffee extract), Japanese radish, fenugreek seed, garden pea, hemp seed, pumpkin seed, oats, potato, dahlia, Stachys species, Strophanthus species, Laminariaceae species (especially Laminaria and Saccharine), Postelsia palmaeformis, Pseudochorda nagaii, Akkesiphycus or Dichapetalum cymosum. The plant extract is preferably enriched in trigonelline, i.e., the starting plant material comprises one or more other compounds in addition to the trigonelline, and the enriched plant material has a ratio of the trigonelline relative to at least one of the one or more other compounds that is higher than the ratio in the starting plant material.

Therefore, some embodiments of the composition comprise plant sources and/or enriched plant sources that provide at least a portion of the trigonelline in the composition.

In a preferred embodiment, the composition comprises enriched fenugreek extract which provides at least about 25-50% trigonelline in the composition. In a more preferred embodiment, the composition comprises enriched fenugreek extract which provides at least about 28-40% trigonelline.

As used herein, a “composition consisting essentially of trigonelline” contains trigonelline and is substantially free or completely free of any additional compound that affects NAD+ production other than the trigonelline. In a particular non-limiting embodiment, the composition consists of the trigonelline and one or more excipients.

In some embodiments, the composition consisting essentially of trigonelline is optionally substantially free or completely free of other NAD+ precursors, such as one or more of trigonelline derivatives; metabolites and pyrolysis products of trigonelline, such as nicotinamide, nicotinamide riboside, 1-methylnicotinamide, 1-methyl-2-pyridone-5-carboxamide (Me2PY), 1-methyl-4-pyridone-5-carboxamide (Me4PY), and alkyl-pyridiniums, such as 1-methyl-pyridinium and 1,4-dimethylpyridinium; nicotinic acid (“niacin”); or L-tryptophan.

As used herein, “substantially free” means that any of the other compound present in the composition is no greater than 1.0 wt. % relative to the amount of trigonelline, preferably no greater than 0.1 wt. % relative to the amount of trigonelline, more preferably no greater than 0.01 wt. % relative to the amount of trigonelline, most preferably no greater than 0.001 wt. % relative to the amount of trigonelline.

### Protein and High Protein

The term “protein” as used herein includes free form amino acids, molecules between 2 and 20 amino acids (referenced herein as “peptides”), and also includes longer chains of amino acids as well. Small peptides, i.e., chains of 2 to 10 amino acids, are suitable for the composition alone or in combination with other proteins. The “free form” of an amino acid is the monomeric form of the amino acid. Suitable amino acids include both natural and non-natural amino acids. The composition can comprise a mixture of one or more types of protein, for example one or more (i) peptides, (ii) longer chains of amino acids, or (iii) free form amino acids; and the mixture is preferably formulated to achieve a desired amino acid profile/content.

At least a portion of the protein can be from animal or plant origin, for example dairy protein such as one or more of milk protein, e.g., milk protein concentrate or milk protein isolate; caseinates or casein, e.g., micellar casein concentrate or micellar casein isolate; or whey protein, e.g., whey protein concentrate or whey protein isolate. Additionally or alternatively, at least a portion of the protein can be plant protein such as one or more of soy protein or pea protein.

Mixtures of these proteins are also suitable, for example mixtures in which casein is the majority of the protein but not the entirety, mixtures in which whey protein is the majority of the protein but not the entirety, mixtures in which pea protein is the majority of the protein but not the entirety, and mixtures in which soy protein is the majority of the protein but not the entirety. In an embodiment, at least 10 wt. % of the protein is whey protein, preferably at least 20 wt. %, and more preferably at least 30 wt. %. In an embodiment, at least 10 wt. % of the protein is casein, preferably at least 20 wt. %, and more preferably at least 30 wt. %. In an embodiment, at least 10 wt. % of the protein is plant protein, preferably at least 20 wt. %, more preferably at least 30 wt. %.

Whey protein may be any whey protein, for example selected from the group consisting of whey protein concentrates, whey protein isolates, whey protein micelles, whey protein hydrolysates, acid whey, sweet whey, modified sweet whey (sweet whey from which the caseino-glycomacropeptide has been removed), a fraction of whey protein, and any combination thereof.

Casein may be obtained from any mammal but is preferably obtained from cow milk and preferably as micellar casein.

The protein may be unhydrolyzed, partially hydrolyzed (i.e., peptides of molecular weight 3 kDa to 10 kDa with an average molecular weight less than 5 kDa) or extensively hydrolyzed (i.e., peptides of which 90% have a molecular weight less than 3 kDa), for example in a range of 5% to 95% hydrolyzed. In some embodiments, the peptide profile of hydrolyzed protein can be within a range of distinct molecular weights. For example, the majority of peptides (>50 molar percent or >50 wt. %) can have a molecular weight within 1-5 kDa, or 5-10 kDa, or 10-20 kDa.

At least a portion of the protein is selected from the group consisting of (i) free form amino acids, (ii) unhydrolyzed protein, (iii) partially hydrolyzed protein, (iv) extensively hydrolyzed protein, and (v) mixtures thereof.

The protein can comprise essential amino acids and/or conditionally essential amino acids, e.g., such amino acids that may be insufficiently delivered due to low caloric intake or illness. For example, the protein can comprise one or more essential amino acids selected from the group consisting of, isoleucine, leucine, valine, histidine, tryptophan, histidine, lysine, methionine, phenylalanine, and threonine; and each of these amino acids (if present) may be administered in the composition in a daily dose from about 0.0476 to about 47.6 mg amino acid/kg bw. Notably, lower intake of methionine leads to lower levels of protein translation and ultimately muscle synthesis.

The protein can comprise one or more conditionally essential amino acids (e.g., amino acids conditionally essential in illness or stress) selected from the group consisting of arginine, cysteine, glutamine, glycine, proline, ornithine, serine and tyrosine; and each of these amino acids (if present) may be administered in the composition in a daily dose from about 0.0476 to about 47.6 mg amino acid/kg bw.

The composition can comprise one or more branched chain amino acids (BCAAs) in at least one form selected from the group consisting of (i) free form, (ii) bound to at least one additional amino acid, and (iii) mixtures thereof. For example, the branched chain amino acids can comprise leucine, isoleucine and/or valine, in free form and/or bound as peptides and/or proteins such as dairy, animal or plant proteins. A daily dose of the branched chain amino acids can include one or more of 0.35-142.85 mg/kg bw Leucine, preferably 0.175-71.425 mg/kg bw Leucine; 0.175-71.425 mg/kg bw Isoleucine or 4-hydroxyisoleucine; and 0.175-71.425 mg/kg bw Valine. The daily dose of the one or more branched chain amino acids can be provided by one or more servings of the composition per day. Such doses are particularly applicable to complete nutrition compositions, but one of ordinary skill will readily recognize how to adapt these doses for an oral nutritional supplement (ONS).

“High protein” refers to a protein content which is at least about 25% of the total caloric intake of a composition and more preferably at least about 36% or greater of the total caloric intake of the composition of the invention.

In grams protein per 100 kcal this means that the composition has a protein/energy ratio at least about 6 g protein/100 kcal, preferably at least about 9 g protein/100 kcal or greater.

As non-limiting examples, the composition can be administered in a daily dose that provides an amount of protein greater than 1.0 g protein/kg body weight/day, preferably greater than 1.2 g protein/kg body weight/day; for example up to 2.5 g protein/kg body weight/day (e.g., 1.0-2.5 g protein/kg body weight/day; 1.2-2.5 g protein/kg body weight/day; or 1.5-2.5 g protein/kg body weight/day), preferably up to 2.0 g protein/kg body weight/day (e.g., 1.0-2.0 g protein/kg body weight/day; 1.2-2.0 g protein/kg body weight/day; or 1.5-2.0 g protein/kg body weight/day), and more preferably up to 1.5 g protein/kg body weight/day (e.g., 1.0-1.5 g protein/kg body weight/day or 1.2-1.5 g protein/kg body weight/day). The daily dose of the protein can be provided by one or more servings of the composition per day.

If the composition is in liquid form, non-limiting examples of suitable high protein concentrations include 6-20 g protein/100 ml, e.g., 6-11 g protein/100 ml; 7-14 g protein/100 ml; 7-12 g protein/100 ml; 8-11 g protein/100 ml, 8-20 g protein/100 ml; 9-20 g protein/100 ml; and 11-20 g protein/100 ml.

### Composition Formulation

The composition can be selected from the group consisting of: a food product, a beverage product, a food supplement, an oral nutritional supplement (ONS), a medical food, and combinations thereof wherein the “high protein” content is at least about 25% of the total caloric intake of the composition and more preferably at least about 36% or greater of the total caloric intake of the composition of the invention.

In an embodiment, at least a portion of the protein is selected from the group consisting of (i) protein from an animal source, (ii) protein from a plant source and (iii) a mixture thereof.

In an embodiment, at least a portion of the protein is selected from the group consisting of (i) milk protein, (ii) whey protein, (iii) caseinate, (iv) micellar casein, (v) pea protein, (vi) soy protein and (vii) mixtures thereof.

In an embodiment, the protein has a formulation selected from the group consisting of (i) at least 50 wt. % of the protein is casein, (ii) at least 50 wt. % of the protein is whey protein, (iii) at least 50 wt. % of the protein is pea protein and (iv) at least 50 wt. % of the protein is soy protein.

In an embodiment, at least a portion of the protein is selected from the group consisting of (i) free form amino acids, (ii) unhydrolyzed protein, (iii) partially hydrolyzed protein, (iv) extensively hydrolyzed protein, and (v) mixtures thereof. The protein can comprise one or more amino acids selected from the group consisting of histidine, isoleucine, leucine, lysine, methionine, phenylalanine, threonine, tryptophan, valine, arginine, cysteine, glutamine, glycine, proline, ornithine, serine, tyrosine, and mixtures thereof. The protein can comprise peptides having a length of 2 to 10 amino acids.

The protein can comprise essential amino acids and/or conditionally essential amino acids, e.g., such amino acids that may be insufficiently delivered in disease or recovery conditions. For example, the protein can comprise one or more essential amino acids selected from the group consisting of Isoleucine, Leucine, Valine, Tryptophan, Histidine, Lysine, Methionine, Phenylalanine, and Threonine; and each of these amino acids, if present, may be administered in the composition in a daily dose from about 0.0476 to about 47.6 mg amino acid/kg body weight. Notably, lower intake of methionine leads to lower levels of protein translation and ultimately muscle synthesis.

In a preferred embodiment, the one or more essential amino acids is selected from the group consisting of: Leucine, Isoleucine, Valine and/or Tryptophan.

In one preferred embodiment, the one or more amino acids is 4-hydroxyisoleucine.

The composition can comprise one or more of Leucine, Isoleucine, Valine or Tryptophan, in free form and/or bound as peptides and/or proteins such as dairy, animal or plant proteins. A daily dose of the composition can include one or more of 0.175-142.85 mg/kg body weight Leucine, preferably 0.35-71.425 mg/kg bw Leucine; 0.175-71.425 mg/kg body weight Isoleucine or 4-hydroxyisoleucine; 24-87 mg/kg body weight Valine; 5-7 mg/kg body weight Tryptophan. The daily dose of the one or more amino acids can be provided by one or more servings of the composition per day.

In an embodiment, the composition comprises branched chain amino acids in at least one form selected from the group consisting of (i) free form, (ii) bound to at least one additional amino acid, and (iii) mixtures thereof. The branched chain amino acids can comprise leucine in an amount effective to activate mTOR in the individual.

In an embodiment, at least a portion of the protein is 5 to 95% hydrolyzed.

In an embodiment, the protein has a formulation selected from the group consisting of (i) at least 50% of the protein has a molecular weight of 1-5 kDa, (ii) at least 50% of the protein has a molecular weight of 5-10 kDa and (iii) at least 50% of the protein has a molecular weight of 10-20 kDa.

### Branched Chain Amino Acids

There are three branched chain amino acids (BCAAs): leucine, isoleucine, and valine which are proteinogenic amino acids. The three proteinogenic BCAAs are among the nine essential amino acids for humans, accounting for 35% of the essential amino acids in muscle proteins. These amino acids help to boost muscle growth and enhance recovery after exercise or trauma.

The average adult should consume a minimum of 34 mg/kg to 144 mg/kg of body weight per day with a total of maximum 15-25 g per day.

### Tryptophan

Tryptophan is also known as (2S)-2-amino-3-(1H-indol-3-yl)propanoic acid or 2-Amino-3-(1H-indol-3-yl)propanoic acid.

Tryptophan is an essential amino acid which cannot be synthesized in the body and must be obtained from the diet. It is absorbed from food and converted into the body into 5-HTP (5-hydroxytryptophan), and then to serotonin, melatonin, and vitamin B6 (nicotinamide).

Tryptophan can significantly impact muscle mass through its metabolite serotonin, and animals deficient in tryptophan are known to show low levels of growth hormone (GH) and significant muscle atrophy, a downstream effect of reduced GH-IGF1 signaling (Dukes, A. et al Nutrition. 2015 July-August; 31(0): 1018-1024).

Tryptophan supplementation may be provided in the form of L-tryptophan, for example, from 250 mg to 1000 mg/day. The recommended daily allowance is approximately 5 mg/kg body weight/day for adults but this dose may be adapted depending on need.

### Creatine

Creatine is produced endogenously in the body from amino acids glycine and arginine, with additionally methionine to catalyze the transformation of guanidinoacetate to creatine in the liver, kidney and pancreas. Creatine or cyclocreatine are substrates for the enzyme creatine kinase which increase phosphocreatine (PCr) or phosphocyclocreatine (PCCr) levels and ATP generation and thereby may exert protective effects on skeletal muscle. Especially under conditions of high energy demand such as intense physical activity, trauma, disease and illness; it may be necessary to supplement creatine as it assists in increasing muscle mass and muscle strength.

Creatine supplementation may be provided in the form of creatine ethyl ester, gluconate, monohydrate, and nitrate forms. Creatine may be provided in micronized, effervescent and serum formulations.

For adults, different unit dosing regimes of creatine may be considered depending on the indication.

For age-related muscle loss, the preferred dosing is to use a short-term “loading dose” followed by a long-term maintenance dose. Loading doses are typically 20 grams daily for 4-7 days.

Maintenance doses are typically 2-10 grams daily. In one preferred embodiment, creatine supplementation should be combined with resistance training.

For athletic performance, the preferred dosing is a short-term “loading dose” followed by a long-term maintenance dose. Loading doses are typically 20 grams daily for 4-7 days. Maintenance doses are typically 2-10 grams daily.

For increasing muscle strength, for example after injury, trauma or surgery; the preferred dosing is a short-term “loading dose” followed by a long-term maintenance dose. The most common loading doses are typically around 20 grams daily for 5-7 days. Maintenance doses ranging from 1 to 27 grams daily have also been used.

For children, with syndromes caused by problems making or transporting creatine, doses of 400-800 mg of creatine per kg of body weight have been taken daily for up to 8 years. Also, 4-8 grams of creatine has been taken daily for up to 25 months.

### Methods of Administration of Composition

A composition of the invention can increase NAD+ in muscle, for example a skeletal muscle. Non-limiting examples of such muscle include one or more of the following: vastus lateralis, gastrocnemius, tibialis, soleus, extensor, digitorum longus (EDL), biceps femoris, semitendinosus, semimembranosus, gluteus maximus, extra-ocular muscles, face muscles or diaphragm.

The individual in need can be an ageing individual, such as an ageing animal or an ageing human. In some embodiments, the individual in need of a composition of the invention is an elderly animal or an elderly human.

For non-human mammals such as rodents, some embodiments comprise administering an amount of the composition that provides 1.0 mg to 1.0 g of the trigonelline/kg of body weight of the non-human mammal, preferably 10 mg to 500 mg of the trigonelline/kg of body weight of the non-human mammal, more preferably 25 mg to 400 mg of the trigonelline/kg of body weight of the mammal, most preferably 50 mg to 300 mg of the trigonelline/kg of body weight of the non-human mammal.

For humans, some embodiments comprise administering an amount of the composition that provides 1.0 mg to 10.0 g of the trigonelline/kg of body weight of the human, preferably 10 mg to 5.0 g of the trigonelline/kg of body weight of the human, more preferably 50 mg to 2.0 g of the trigonelline/kg of body weight of the human, most preferably 100 mg to 1.0 g of the trigonelline/kg of body weight of the human.

In some embodiments of the invention, in addition to trigonelline, high protein, creatine and/or tryptophan; the composition may contain additional components such as carbohydrates or fats.

In one embodiment, the composition may include a source of carbohydrates. Any suitable carbohydrate may be used in the composition including, but not limited to, starch (e.g., modified starch, amylose starch, tapioca starch, corn starch), sucrose, lactose, glucose, fructose, corn syrup solids, maltodextrin, xylitol, sorbitol or combinations thereof.

The source of carbohydrates is preferably not greater than 50 energy % of the composition, more preferably not greater than 36 energy % of the composition, and most preferably not greater than 30 energy % of the composition. The composition can have a high protein:carbohydrate energy ratio, for example greater than 0.66, preferably greater than 0.9 and more preferably greater than 1.2.

In an embodiment, the composition may include a source of fat. The source of fat may include any suitable fat or fat mixture. Non-limiting examples of suitable fat sources include vegetable fat, such as olive oil, corn oil, sunflower oil, high-oleic sunflower, rapeseed oil, canola oil, hazelnut oil, soy oil, palm oil, coconut oil, blackcurrant seed oil, borage oil, lecithins, and the like, animal fats such as milk fat; or combinations thereof.

The composition of the invention can be administered to an individual such as a human, e.g., an ageing individual or a critically ill individual, in a therapeutically effective dose. The therapeutically effective dose can be determined by the person skilled in the art and will depend on a number of factors known to those of skill in the art, such as the severity of the condition and the weight and general state of the individual.

The composition is preferably administered to the individual at least two days per week, more preferably at least three days per week, most preferably all seven days of the week; for at least one week, at least one month, at least two months, at least three months, at least six months, or even longer. In some embodiments, the composition is administered to the individual consecutively for a number of days, for example at least until a therapeutic effect is achieved. In an embodiment, the composition can be administered to the individual daily for at least 30, 60 or 90 consecutive days.

The above examples of administration do not require continuous daily administration with no interruptions. Instead, there may be some short breaks in the administration, such as a break of two to four days during the period of administration. The ideal duration of the administration of the composition can be determined by those of skill in the art.

In a preferred embodiment, the composition is administered to the individual orally or enterally (e.g. tube feeding). For example, the composition can be administered to the individual as a beverage, a capsule, a tablet, a powder or a suspension.

The composition can be any kind of composition that is suitable for human and/or animal consumption. For example, the composition may be selected from the group consisting of food compositions, dietary supplements, nutritional compositions, nutraceuticals, powdered nutritional products to be reconstituted in water or milk before consumption, food additives, medicaments, beverages and drinks. In an embodiment, the composition is an oral nutritional supplement (ONS), a complete nutritional formula, a pharmaceutical, a medical or a food product. In a preferred embodiment, the composition is administered to the individual as a beverage. The composition may be stored in a sachet as a powder and then suspended in a liquid such as water for use.

In some instances where oral or enteral administration is not possible or not advised, the composition may also be administered parenterally.

In some embodiments, the composition is administered to the individual in a single dosage form, i.e. all compounds are present in one product to be given to an individual in combination with a meal. In other embodiments, the composition is co-administered in separate dosage forms, for example at least one component separately from one or more of the other components of the composition.

These methods can consist essentially of administering the composition consisting essentially of trigonelline or consisting essentially of trigonelline and high protein or consisting essentially of trigonelline, high protein and creatine or consisting essentially of trigonelline, high protein, creatine and/or tryptophan. As used herein, a “method consisting essentially of administering the composition consisting essentially of trigonelline or consisting of trigonelline” means that any additional compound that affects NAD⁺ production other than the trigonelline is not administered within one hour as the administration of the trigonelline, preferably not administered within two hours as the administration of the trigonelline, more preferably not administered within three hours as the administration of the trigonelline, most preferably not administered in the same day as the administration of the trigonelline. Non-limiting examples of compounds that optionally can be excluded from the method include those disclosed above regarding exclusion from the composition itself.

In each of the compositions and methods disclosed herein, the composition is preferably a food product, including food additives, food ingredients, functional foods, dietary supplements, medical foods, nutraceuticals, oral nutritional supplements (ONS) or food supplements.

The composition can be administered at least one day per week, preferably at least two days per week, more preferably at least three or four days per week (e.g., every other day), most preferably at least five days per week, six days per week, or seven days per week. The time period of administration can be at least one week, preferably at least one month, more preferably at least two months, most preferably at least three months, for example at least four months. In some embodiments, dosing is at least daily; for example, a subject may receive one or more doses daily, in an embodiment a plurality of doses per day. In some embodiments, the administration continues for the remaining life of the individual. In other embodiments, the administration occurs until no detectable symptoms of the medical condition remain. In specific embodiments, the administration occurs until a detectable improvement of at least one symptom occurs and, in further cases, continues to remain ameliorated.

The compositions disclosed herein may be administered to the subject enterally, e.g., orally, or parenterally. Non-limiting examples of parenteral administration include intravenously, intramuscularly, intraperitoneally, subcutaneously, intraarticularly, intrasynovially, intraocularly, intrathecally, topically, and inhalation. As such, non-limiting examples of the form of the composition include natural foods, processed foods, natural juices, concentrates and extracts, injectable solutions, microcapsules, nano-capsules, liposomes, plasters, inhalation forms, nose sprays, nosedrops, eyedrops, sublingual tablets, and sustained-release preparations.

The compositions disclosed herein can use any of a variety of formulations for therapeutic administration. More particularly, pharmaceutical compositions can comprise appropriate pharmaceutically acceptable carriers or diluents and may be formulated into preparations in solid, semi-solid, liquid or gaseous forms, such as tablets, capsules, powders, granules, ointments, solutions, suppositories, injections, inhalants, gels, microspheres, and aerosols. As such, administration of the composition can be achieved in various ways, including oral, buccal, rectal, parenteral, intraperitoneal, intradermal, transdermal, and intratracheal administration. The active agent may be systemic after administration or may be localized by the use of regional administration, intramural administration, or use of an implant that acts to retain the active dose at the site of implantation.

In pharmaceutical dosage forms, the compounds may be administered as their pharmaceutically acceptable salts. They may also be used in appropriate association with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose functional derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional, additives such as solubilizers, isotonic agents, suspending agents, emulsifying agents, stabilizers and preservatives.

The compounds can be utilized in an aerosol formulation to be administered by inhalation. For example, the compounds can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds can be administered rectally by a suppository. The suppository can include a vehicle such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition. Similarly, unit dosage forms for injection or intravenous administration may comprise the compounds in a composition as a solution in sterile water, normal saline or another pharmaceutically acceptable carrier, wherein each dosage unit, for example, mL or L, contains a predetermined amount of the composition containing one or more of the compounds.

Compositions intended for a non-human animal include food compositions to supply the necessary dietary requirements for an animal, animal treats (e.g., biscuits), and/or dietary supplements. The compositions may be a dry composition (e.g., kibble), semi-moist composition, wet composition, or any mixture thereof. In one embodiment, the composition is a dietary supplement such as a gravy, drinking water, beverage, yogurt, powder, granule, paste, suspension, chew, morsel, treat, snack, pellet, pill, capsule, tablet, or any other suitable delivery form. The dietary supplement can comprise a high concentration of the UFA and NORC, and B vitamins and antioxidants. This permits the supplement to be administered to the animal in small amounts, or in the alternative, can be diluted before administration to an animal. The dietary supplement may require admixing, or can be admixed with water or other diluent prior to administration to the animal.

“Pet food” or “pet treat compositions” comprise from about 15% to about 50% crude protein. The crude protein material may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include pork, lamb, equine, poultry, fish, and mixtures thereof. The compositions may further comprise from about 5% to about 40% fat. The compositions may further comprise a source of carbohydrate. The compositions may comprise from about 15% to about 60% carbohydrate. Examples of such carbohydrates include grains or cereals such as rice, corn, milo, sorghum, alfalfa, barley, soybeans, canola, oats, wheat, and mixtures thereof. The compositions may also optionally comprise other materials such as dried whey and other dairy by-products.

In some embodiments, the ash content of the pet food composition ranges from less than 1% to about 15%, and in one aspect, from about 5% to about 10%.

The moisture content can vary depending on the nature of the pet food composition. In a one embodiment, the composition can be a complete and nutritionally balanced pet food. In this embodiment, the pet food may be a “wet food”, “dry food”, or food of intermediate moisture content. “Wet food” describes pet food that is typically sold in cans or foil bags, and has a moisture content typically in the range of about 70% to about 90%. “Dry food” describes pet food which is of a similar composition to wet food, but contains a limited moisture content, typically in the range of about 5% to about 15% or 20%, and therefore is presented, for example, as small biscuit-like kibbles. In one embodiment, the compositions have moisture content from about 5% to about 20%. Dry food products include a variety of foods of various moisture contents, such that they are relatively shelf-stable and resistant to microbial or fungal deterioration or contamination. Also included are dry food compositions which are extruded food products, such as pet foods, or snack foods for companion animals.

### Uses of the Composition

Method and uses of the composition are provided for increasing NAD+ in a subject by administering an effective amount of a composition in an effect unit dose form to prevent and/or treat skeletal muscle diseases or conditions.

In some embodiments, methods and uses of the composition are provide for prevention or treatment of skeletal muscle diseases or conditions. In some embodiments, methods and uses of the composition are for skeletal muscle diseases or conditions such as: sarcopenia, cachexia or precachexia, myopathy, dystrophy, and/or recovery after intense exercise, muscle injury or surgery.

In one embodiment, a composition of the invention is used for preventing and/or treating skeletal muscle disease or conditions in a subject in need comprising the steps of:

i) providing the subject a composition consisting essentially of or consisting of trigonelline and high protein; and
ii) administering the composition to said subject.

In one embodiment, a composition of the invention is used for preventing and/or treating skeletal muscle disease or conditions in a subject in need comprising the steps of:

i) providing the subject a composition consisting essentially of or consisting of trigonelline, high protein and creatine; and
ii) administering the composition to said subject.

In one embodiment, a composition of the invention is used for preventing and/or treating skeletal muscle disease or conditions in a subject in need comprising the steps of:

i) providing the subject a composition consisting essentially of or consisting of trigonelline, high protein wherein said protein comprises branched chain amino acids and creatine; and
ii) administering the composition to said subject.

In one embodiment, a composition of the invention is used for preventing and/or treating skeletal muscle disease or conditions in a subject in need comprising the steps of:

i) providing the subject a composition consisting essentially of or consisting of trigonelline, high protein, creatine and tryptophan; and
ii) administering the composition to said subject.

In some embodiments, the subject is selected from the group consisting of: human, dog, cat, cow, horse, pig, or sheep. The subject is preferably a human in need of prevention or treatment of diseases or conditions affecting skeletal muscle.

### EXAMPLES

### Example 1—Enzymatic Quantification of NAD+ Concentration in Human and Zebrafish after Treatment with Trigonelline

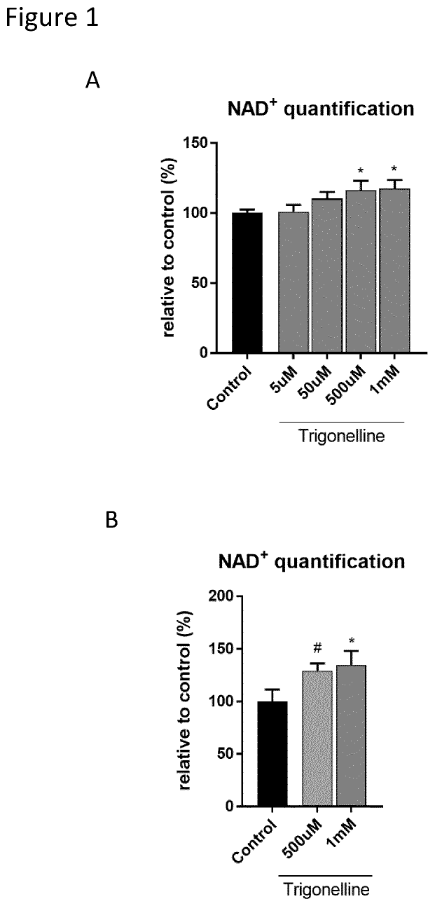

Human primary myoblasts were seeded in 384 well plates at a density of 3′000 cells per well in skeletal muscle growth medium (SKM-M, AMSbio). After one day, the differentiation was induced by a medium change for 4 days using differentiation culture medium (Gibco No. 31330-028). Cells were treated with trigonelline (sigma #T5509) for 6 h. NAD was measured using bioluminescent assay (Promega NAD/NADH-Glo™ #G9071). This is shown in FIG. 1A.

Embryos from wild type zebrafish have been raised at 28° C. under standard laboratory conditions and have been raised at 96 h post-fertilization in 6 well plates (n=20-25). Larvae were treated with trigonelline (sigma #T5509) for 16 h. NAD was measured using colorimetric NAD quantification assay (Biovision NAD/NADH Quantitation Colorimetric Kit #k337-100). This is shown in FIG. 1B.

### Example 2Human Myoblast Differentiation Enhanced by Trigonelline

Human primary myoblasts from two different donors were seeded in 6 well plates at a density of 200,000 cells per well in skeletal muscle growth medium (SKM-M, AMSbio). After one day, the differentiation was induced by a medium change for 4 days using differentiation culture medium (Gibco No. 31330-028). Cells were treated with isotopically labelled trigonelline (¹³C carbonyl; 3²H on methyl) for 6 h.

Cell extracts were separated on a Vanquish UHPLC+focused LC system (Thermo Scientific) with a hydrophilic liquid chromatography (HILIC) iHILIC-Fusion(P) column (Hilicon) carrying the dimensions 150×2.1 mm, 5 μm and a guard column (iHILIC-fusion(P), Hilicon) in front. The separation of metabolites was achieved by applying a linear solvent gradient in normal phase at 0.25 mL/min flow rate and 35° C. of temperature. As mobile phase, solvent A was water with 10 mM ammonium acetate and 0.04% (v/v) ammonium hydroxide, pH ˜9.3, and solvent B was acetonitrile.

The eluting metabolites were analyzed with an Orbitrap Fusion Lumos mass spectrometer (Thermo Scientific) with a heated electrospray ionization (H-ESI) source in positive and negative mode at a resolution of 60,000 at m/z of 200. Instrument control and data analysis were conducted using Xcalibur (Thermo Scientific).

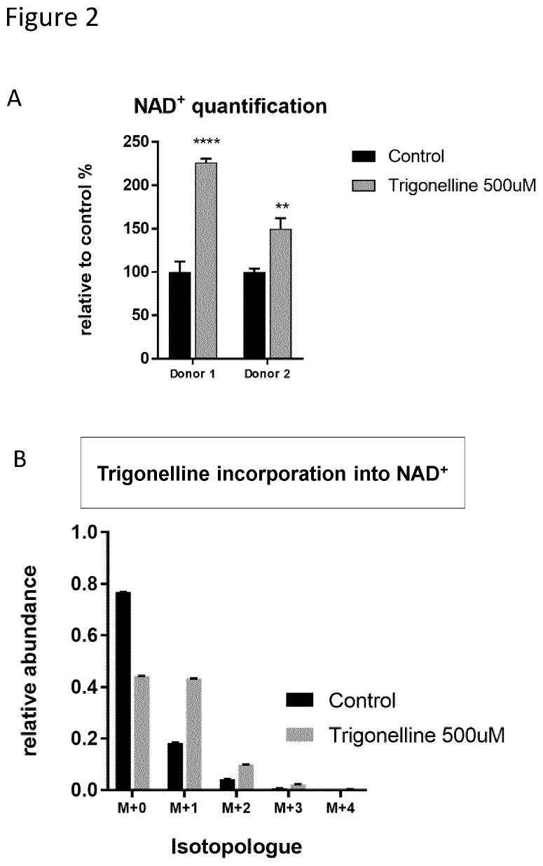

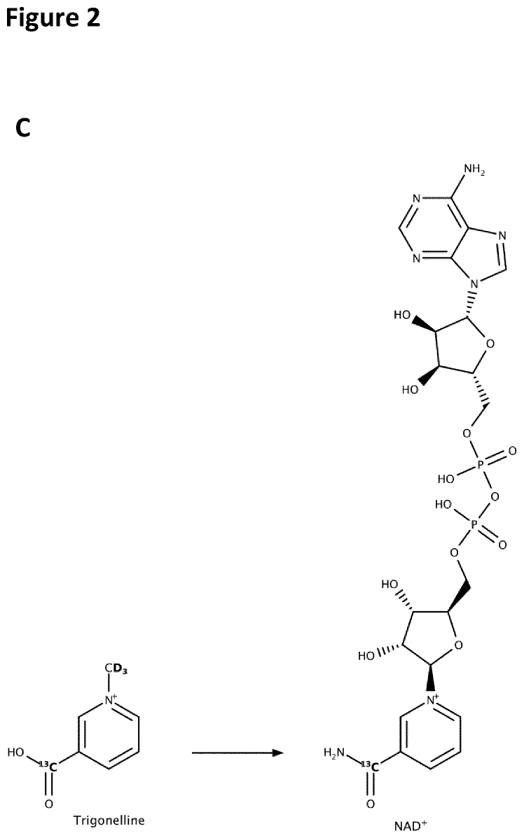

FIG. 2A shows the enhancement of NAD+ with trigonelline given at 500 μm. FIG. 2B shows the increase in relative abundance of labelled NAD+ (M+1) after treatment with labelled trigonelline, the dose of 500 μm compared to the control which is the naturally occurring NAD+ in differentiated primary myoblasts.

### Example 3—Liver and Muscle NAD+ Concentration after Oral or Intraperitoneal Administration of Trigonelline

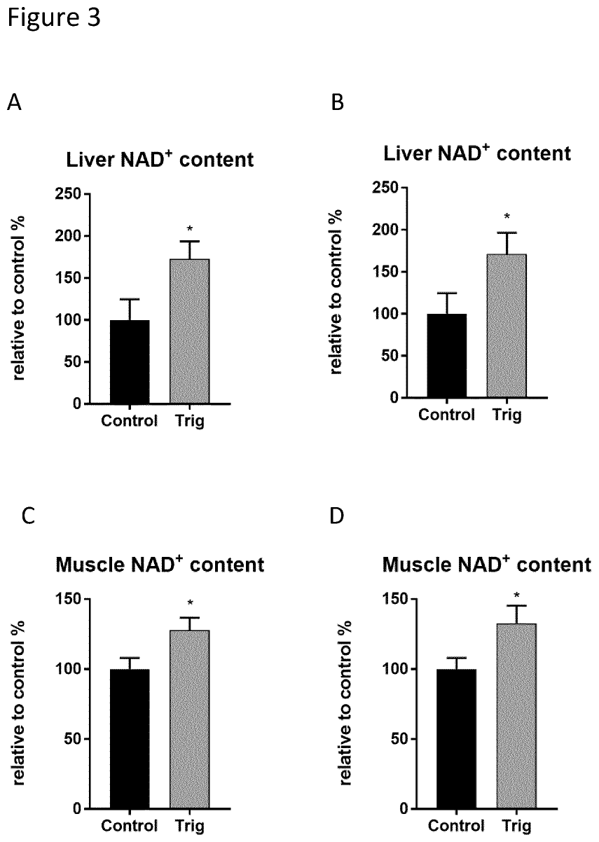

10 weeks C57BL/6JRj male mice were fed a diet (Safe 150) and then received oral gavage or intraperitoneal injection of trigonelline (250 mg/kg, n=5/group). Tissues were harvested and flash frozen in liquid nitrogen after 120 minutes of treatment. NAD was measured in gastrocnemius muscle and in liver using colorimetric NAD quantification assay (Biovision NAD/NADH Quantitation Colorimetric Kit #k337-100). FIG. 3 shows the enzymatic quantification of NAD+ in mice 120 minutes after receiving 250 mg/kg trigonelline by oral gavage (FIGS. 3A, 3C) or intraperitoneal administration (FIGS. 3B, 3D).

### Example 4: NAD⁺ Measured in Human Primary Myoplasts after Treatment with Chemically Synthesized Trigonelline or Fenugreek Seed Extract Enriched in Trigonelline

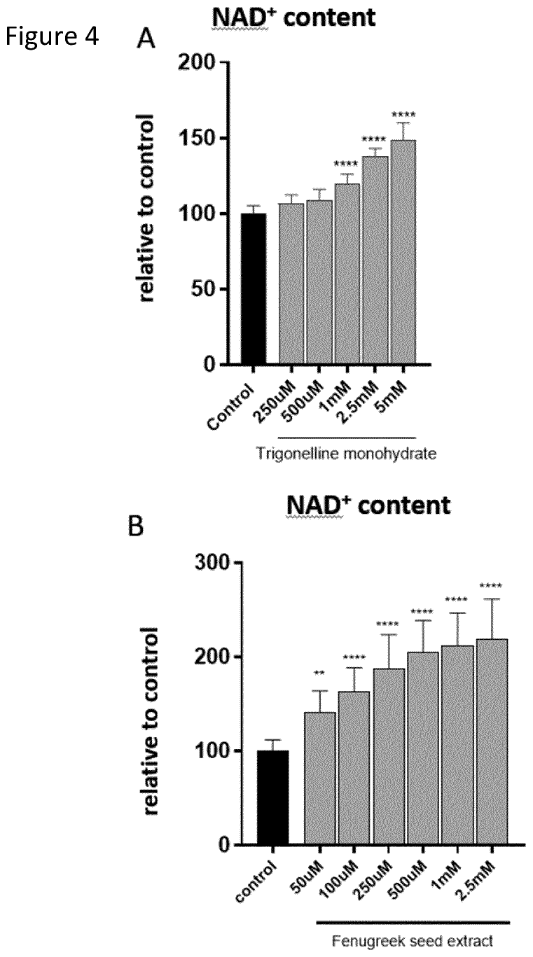

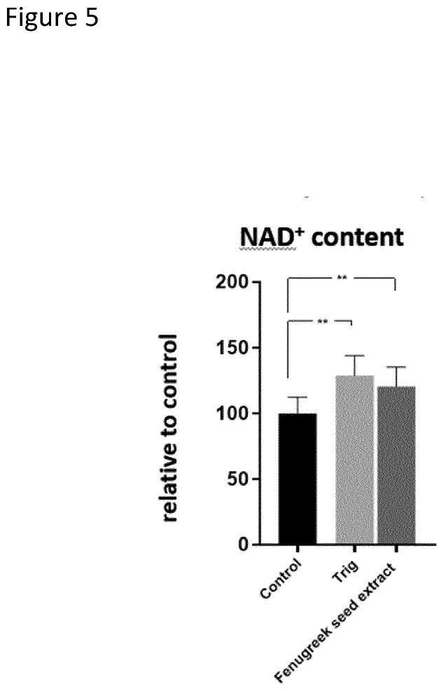

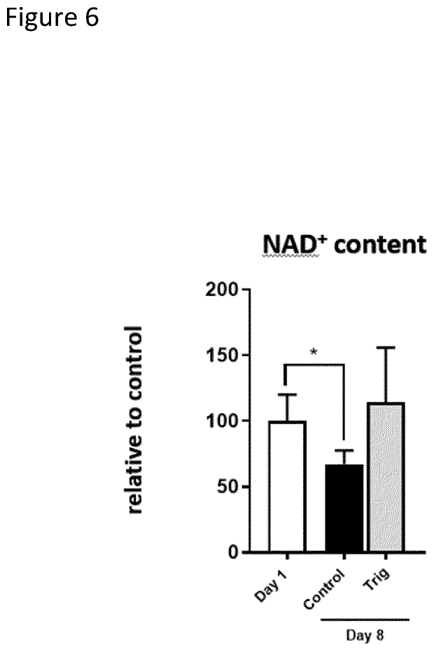

Human primary myoblasts were seeded in 96 well plates at a density of 12′000 cells per well in skeletal muscle growth medium (SKM-M, AMSbio). After one day, the differentiation is induced by a medium change for 4 days. Cells were treated with synthetic trigonelline monohydrate (FIG. 4A) or with Fenugreek seed extract enriched in trigonelline containing 40.45% trigonelline (FIG. 4B) for 16 h at difference doses. NAD⁺ was measured using colorimetric NAD⁺ quantification assay (Biovision NAD⁺/NADH Quantitation Colorimetric Kit #k337-100).

This experiment demonstrated that both the chemically synthesized trigonelline and the trigonelline from the Fenugreek seed extract showed a significant increase in NAD⁺ content compared to the control. For the Fenugreek seed extract, it was more potent at lower doses than the chemically synthesized trigonelline.

### Example 5: NAD⁺ Measured in Mouse Liver after Treatment with Chemically Synthesized Trigonelline or Fenugreek Seed Extract Enriched in Trigonelline

10 weeks C57BL/6JRj male mice received trigonelline (sigma #T5509) or fenugreek seed extract enriched in trigonelline (40.45% trigonelline) by oral gavage (equimolar of 300 mg/kg trigonelline, n=8/group). After 120 minutes treatment, the liver was harvested and flash frozen in liquid nitrogen. NAD⁺ was measured in liver using an enzymatic method adapted from Dall, M., et al., Mol Cell Endocrinol, 2018. 473: p. 245-256.

This experiment demonstrated that both the chemically synthesized trigonelline and the trigonelline from the Fenugreek seed extract showed a significant increase in NAD⁺ content in the liver compared to the control.

### Example 6: Tests in C. elegans to Measure Survival, Speed, Mobility and Stimulated Mobility

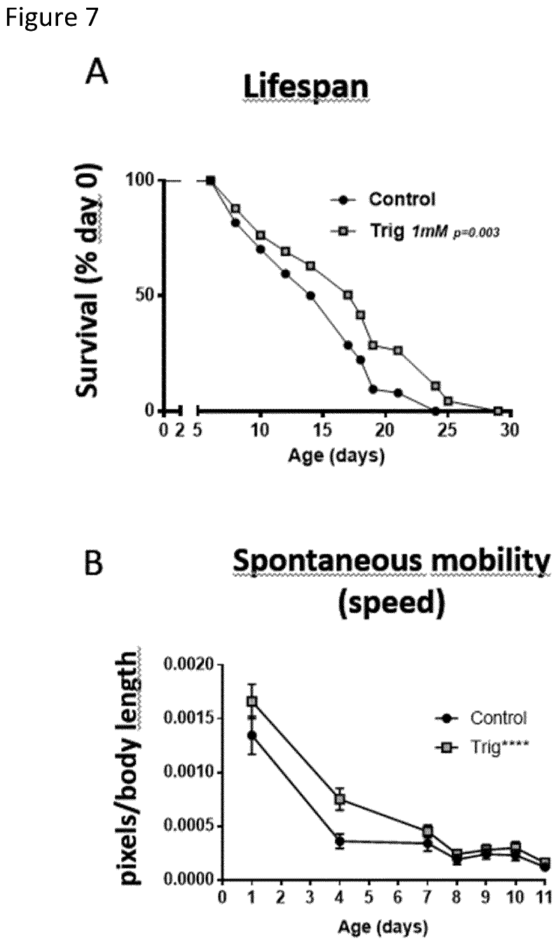

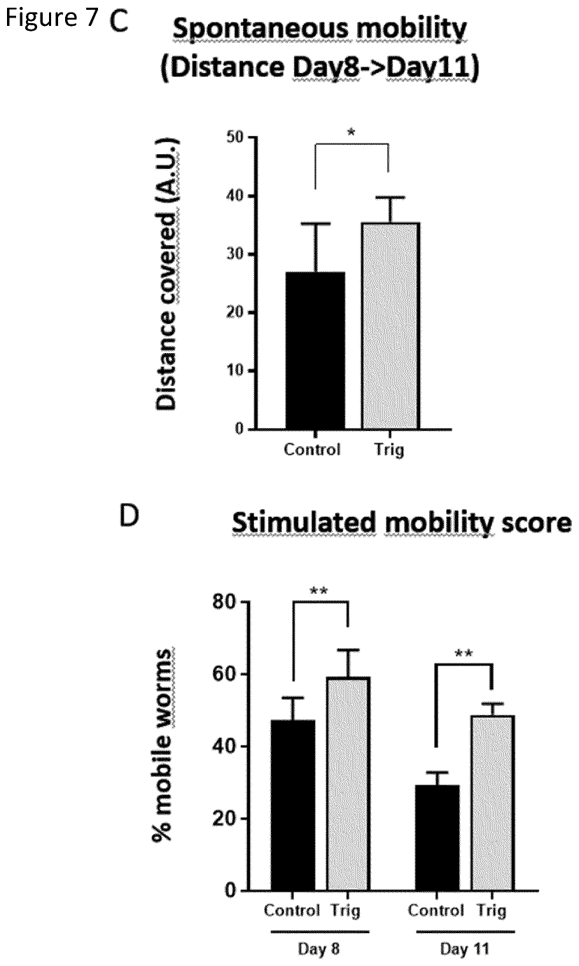

Worm lifespan tests were performed using about 100 animals per condition and scored manually every other day. Trigonelline treatment and experimental measurements were started at Day 1 of wild type N2 worm adulthood, in a regimen of chronic exposure till experiments termination. FIG. 7A demonstrates the mean survival of the worms in days comparing the control to the trigonelline treated worms with the trigonelline treated worms. Survival curve of C elegans treated with 1 mM trigonelline chloride increases lifespan by 21%.

C. elegans mobility test was performed using the Movement Tracker software (Mouchiroud, L. et al. Curr Protoc Neurosci 77, 8.37.1-8.37.21 (2016)). The experiments were repeated at least twice. Trigonelline treatment and experimental measurements were started at Day 1 of wild type N2 worm adulthood, in a regimen of chronic exposure till experiments termination.

FIG. 7B measured the mean speed measured during spontaneous mobility assay performed from day 1 adulthood in 1 mM trigonelline chloride treated worms compared to controls. C. elegans treated with 1 mM trigonelline chloride increased the mean speed compared to the control.

FIG. 7C showed that the distance travelled during the spontaneous mobility assay in advanced aging phase was significantly increased in C. elegans treated with 1 mM trigonelline chloride compared to control.

45 to 60 worms per condition were manually scored for mobility after poking. Worms that were unable to respond to any repeated stimulation were scored as dead. Results were representative of data obtained from at least two independent experiments. Trigonelline treatment and experimental measurements were started at Day 1 of wild type N2 worm adulthood, in a regimen of chronic exposure till experiments termination.

FIG. 7D showed that the stimulated mobility score assessed for day 8 and day 11 old worms indicated that C. elegans treated with 1 mM trigonelline chloride were more responsive to a physical stimulus than the control.

*,** indicate difference from the control, Student test, with p<0.05, p<0.01, respectively.

### Example 7: Structural Integrity of Myofibrils and Myosin Improved with Treatment Using Trigonelline

Age-related morphological changes in myosin structure are typically observed in high-salt ATPase activities of myofibrils and myosin wherein the myofibril structure becomes less organized with advanced age.

RW1596 (myo-3p::GFP) worms were collected at Day 1 (young adults) and at Day 11 (aged animals) for muscle integrity assessment. Worms were immobilized with tetramisole and analyzed by confocal microscopy, to assess the muscle fibers morphology shown by GFP fluorescence imaging. Trigonelline treatment with 1 mM trigonelline chloride and experimental measurements were started at Day 1 of wild type N2 worm adulthood, in a regimen of chronic exposure till experiments termination.

Upon examination of the morphological structure of using fluorescence microscopy of GFP-tagged myosin, we were able to see an improved more organized myofibrillar structure with the trigonelline treated 11 day old worms compared to the age matched control worms.

### Example 8: Ratio of Mitochondrial to Nuclear DNA in Control and Trigonelline Treated C. elegans

Absolute quantification of the mtDNA copy number in wild type N2 worms was performed by real-time PCR. Relative values for nduo-1, and act-1 were compared within each sample to generate a ratio representing the relative level of mitochondrial DNA per nuclear genome. The average of at least two technical repeats was used for each biological data point. Each experiment was performed on at least ten independent biological samples (individual worms). Trigonelline treatment with 1 mM trigonelline chloride and experimental measurements were started at Day 1 of wild type N2 worm adulthood, in a regimen of chronic exposure till experiments termination.

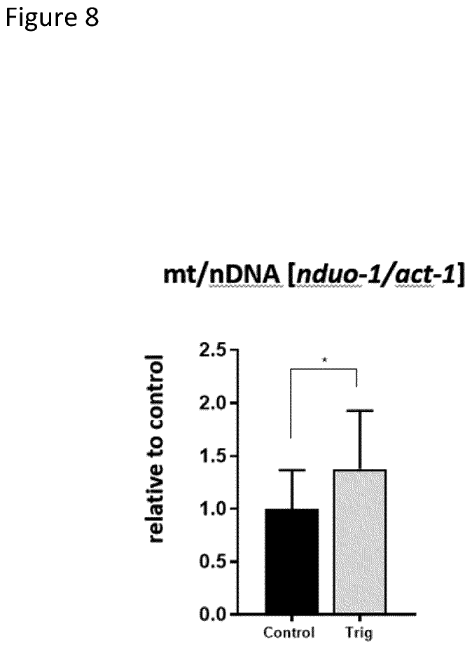

FIG. 8 shows the ratio of a mitochondrial-encoded gene (nduo-1) represented as relative to a nuclear-encoded gene (act-1) in day 8 old worms. *indicate difference from the control, Student test, with p<0.05. Data are presented as Mean+/−SD

In the trigonelline treated group, the mitrochondrial expression relative to the nuclear expression was higher than in the control group.

1. A method for preventing or treating skeletal muscle disease or conditions comprising administering to a subject in need of same a composition comprising trigonelline and high protein for increasing NAD+ levels in skeletal muscle.

2. Method according to claim 1 wherein the composition comprises creatine.

3. Method according to claim 1, and wherein the composition comprises tryptophan.

4. Method according to claim 1 wherein the composition is selected from the group consisting: of a food product, beverage product, a food supplement, an oral nutritional supplement (ONS), a medical food, and combinations thereof.

5. Method according to claim 1 wherein the trigonelline is selected from an extract of coffee, fenugreek, hemp or algae.

6. Method according to claim 1 wherein trigonelline is selected from an extract of fenugreek which contains at least about 25%-50% trigonelline.

7. Method according to claim 1 wherein trigonelline is chemically synthesized and which contains at least about 90% trigonelline.

8. Method according to claim 1 wherein the high protein is at least 25% of the total energy of the composition.

9. Method according to claim 1 wherein the composition has a protein/energy ratio at least about 6 g protein/100 kcal.

10. Method according to claim 1 wherein the high protein comprises branched chain amino acids is selected from the group consisting of: leucine, isoleucine and/or valine.

11. Method according to claim 1 wherein the high protein is 4-hydroxyisoleucine.

12. Method according to claim 1 wherein the protein is selected from the group consisting of: (i) milk protein, (ii) whey protein, (iii) caseinate, (iv) micellar casein, (v) pea protein, (vi) soy protein and (vii) mixtures thereof.

13. Method according to claim 2 wherein the creatine is selected from the group consisting of: creatine ethyl ester, creatine gluconate, creatine monohydrate, and creatine nitrate forms.

14. Method according to claim 1 to maintain or increase skeletal muscle function in a subject.

15-17. (canceled)

18. Method according to claim 1 to enhance recovery of skeletal muscle after intense exercise.

19. Method according to claim 1 to enhance recovery of skeletal muscle after injury, trauma or surgery.

20. Method according to claim 1 to enhance recovery of skeletal muscle after skeletal muscle disease and/or conditions.

21. Method according to claim 20 wherein skeletal muscle disease and/or condition is selected from the group consisting of: sarcopenia, cachexia or precachexia, myopathy, dystrophy, and/or recovery after intense exercise, muscle injury and surgery.

22. Method according to claim 21 wherein cachexia is associated with a disease selected from cancer, chronic heart failure, renal failure, chronic obstructive pulmonary disease, AIDS, autoimmune disorders, chronic inflammatory disorders, cirrhosis of the liver, anorexia, chronic pancreatitis, metabolic acidosis and neurodegenerative disease.

23-30. (canceled)
